(19) United States
(12) Patent Application Publication
(10) Pub. No.: US 2026/0062750 A1
(43) Pub. Date: Mar. 5, 2026

(54) DIAGNOSIS AND TREATMENT FOR CARDIAC CONDITIONS BASED ON SEQUENCING DATA FOR LPAGENE

(71) Applicant: Helix, Inc., San Mateo, CA (US)

(72) Inventors: Kelly Marie Schiabor Barrett, San Diego, CA (US); Hang Dai, Mountain View, CA (US)

(21) Appl. No.: 19/002,284

(22) Filed: Dec. 26, 2024

Related U.S. Application Data
(60) Provisional application No. 63/691,067, filed on Sep. 5, 2024.

(51) Int. Cl. C12Q 1/6883; G16B 30/10

(52) U.S. Cl. CPC C12Q 1/6883; G16B 30/10; C12Q 2600/106

(57) ABSTRACT

Systems and methods are provided for selecting a patient for intervention relating to genetically predicted lipoprotein (a) (“Lp(a)”) levels. A method may obtain or having obtained a biological sample from the patient. A method may perform or having performed sequencing on the biological sample, comprising. A method may acquire reads for the patient. A method may mask at least one portion of a gene LPA at a reference genome to facilitate alignment of the reads to a reference genome. A method may determine a pseudocount of copy number within the gene LPA at a genome of the patient. A method may in an event that the pseudocount is not an expected amount, selecting the patient for the intervention. A method may in an event that the pseudocount is an expected amount, omitting selection of the patient for the intervention.

### TECHNICAL FIELD

The disclosure relates to the field of genomic analysis, and in particular, to determining suitability for treatment of cardiac conditions, based on genetic data describing the gene LPA.

### BACKGROUND

Lipoprotein (a), generally referred to as “Lp(a)” is a type of particle comprising both protein and lipid components. High levels of Lp(a) in the blood are associated with a greater incidence of various cardiovascular conditions. However, the test for directly measuring Lp(a) in blood is expensive, difficult to perform, and requires specialized equipment.

Studies indicate that Copy Number Variants (CNVs) within the gene LPA that encodes apolipoprotein(a), a component of Lp(a) that has an impact upon Lp(a) in the bloodstream. Presently, long-read sequencing equipment (i.e., sequencing equipment that generates reads longer than 1 kilobase (kb) in length) and/or gel electrophoresis is used to attempt to determine the number, nature, and location of CNVs in LPA (i.e., across both chromosomal copies). Unfortunately, these techniques are not wholly precise. They also exhibit many of the same deficiencies of directly testing Lp(a) levels in blood. Specifically, long-read sequencing techniques and gel electrophoresis are expensive, difficult to perform, and require specialized equipment. Because of this, sequencing-based insights are rarely leveraged to facilitate Lp(a) related diagnostics, and Lp(a)-related cardiac conditions remain underdiagnosed.

Hence, scientists and medical practitioners continue to seek out enhanced systems and methods for acquiring insights into Lp(a) levels in a manner that is both cost-effective and accurate.

### SUMMARY OF THE DISCLOSURE

In some aspects, the techniques described herein relate to a method for selecting a patient for intervention relating to genetically predicted lipoprotein (a) (“Lp(a)”) levels, the method including: obtaining or having obtained a biological sample from the patient; performing or having performed sequencing on the biological sample, including: acquiring reads for the patient; and masking at least one portion of a gene LPA at a reference genome to facilitate alignment of the reads to a reference genome; determining a pseudocount of copy number within the gene LPA at a genome of the patient; in an event that the pseudocount is not an expected amount, selecting the patient for the intervention; and in an event that the pseudocount is an expected amount, omitting selection of the patient for the intervention.

In some aspects, the techniques described herein relate to a non-transitory computer readable medium embodying programmed instructions which, when executed by a processor, are operable for performing a method for selecting a patient for intervention relating to genetically predicted lipoprotein (a) (“Lp(a)”) levels, the method including: obtaining or having obtained a biological sample from the patient; performing or having performed sequencing on the biological sample, including: acquiring reads for the patient; and masking at least one portion of a gene LPA at a reference genome to facilitate alignment of the reads to a reference genome; determining a pseudocount of copy number within the gene LPA at a genome of the patient; in an event that the pseudocount is not an expected amount, selecting the patient for the intervention; and in an event that the pseudocount is an expected amount, omitting selection of the patient for the intervention.

In some aspects, the techniques described herein relate to a system for selecting a patient for intervention relating to genetically predicted lipoprotein (a) (“Lp(a)”) levels, the system including: a genomics server, including: an interface configured to acquire reads for a patient; and a controller configured to acquire reads for the patient, and mask at least one portion of a gene LPA at a reference genome to facilitate alignment of the reads to a reference genome, the controller further configured to determine a pseudocount of copy number within the gene LPA at a genome of the patient; and in an event that the pseudocount is not an expected amount, the controller is configured to select the patient for the intervention; and in an event that the pseudocount is an expected amount, the controller is configured to omit selection of the patient for the intervention.

Other illustrative embodiments (e.g., methods and computer-readable media relating to the foregoing embodiments) may be described below. The features, functions, and advantages that have been discussed can be achieved independently in various embodiments or may be combined in yet other embodiments, further details of which can be seen with reference to the following description and drawings.

### BRIEF DESCRIPTION OF THE DRAWINGS

Some embodiments of the present disclosure are now described, by way of example only, and with reference to the accompanying drawings. The same reference number represents the same element or the same type of element on all drawings.

FIG. 1 is a diagram depicting a sample processing architecture in an illustrative embodiment.

FIG. 2 is a block diagram illustrating a genomics architecture in an illustrative embodiment.

FIG. 3 is a flowchart depicting a method of selectively treating a patient based on a pseudocount of copy number within LPA for the patient.

FIG. 4 is a flowchart depicting a method for determining pseudocounts in an illustrative embodiment.

FIG. 5 is a flowchart depicting a method for multi-factor evaluation of genetic risk related to Lp(a) in an illustrative embodiment.

FIG. 6 is a graph that depicts correlations between pseudocounts and measured Lp(a) levels in an illustrative embodiment.

FIG. 7 is a table that summarizes sequencing data for the gene LPA for individuals in an illustrative embodiment.

FIG. 8 is a table that summarizes variant data for the gene LPA for individuals in an illustrative embodiment.

FIG. 9 is a table that summarizes biomarker test data for individuals in an illustrative embodiment.

FIGS. 10-11 depict Graphical User Interfaces (GUIs) that facilitate acquisition of LPA risk status, and/or tests for follow-up diagnosis and treatment for a patient having a high LPA risk status, in illustrative embodiments.

FIG. 12 depicts an illustrative computing system operable to execute programmed instructions embodied on a computer readable medium.

### DETAILED DESCRIPTION

The figures and the following description depict specific illustrative embodiments of the disclosure. It will thus be appreciated that those skilled in the art will be able to devise various arrangements that, although not explicitly described or shown herein, embody the principles of the disclosure and are included within the scope of the disclosure. Furthermore, any examples described herein are intended to aid in understanding the principles of the disclosure, and are to be construed as being without limitation to such specifically recited examples and conditions. As a result, the disclosure is not limited to the specific embodiments or examples described below, but by the claims and their equivalents.

Embodiments described herein beneficially utilize masking for a sequencing technology (e.g., short-read technology) in order to generate a pseudocount of CNVs within LPA for a patient. For example, masking may be performed to facilitate alignment of reads to the KIV-2 region of an LPA reference sequence. In embodiments where patients are sequenced using the same assay, coverage normalization may facilitate consistent measurement of pseudocounts across an entire population of individuals. The pseudocount therefore provides quantitative insights into whether, and to what degree, a patient has the same or a different number of CNVs (e.g., copies of KIV-2) than is typical for the population.

### Processing Architecture.

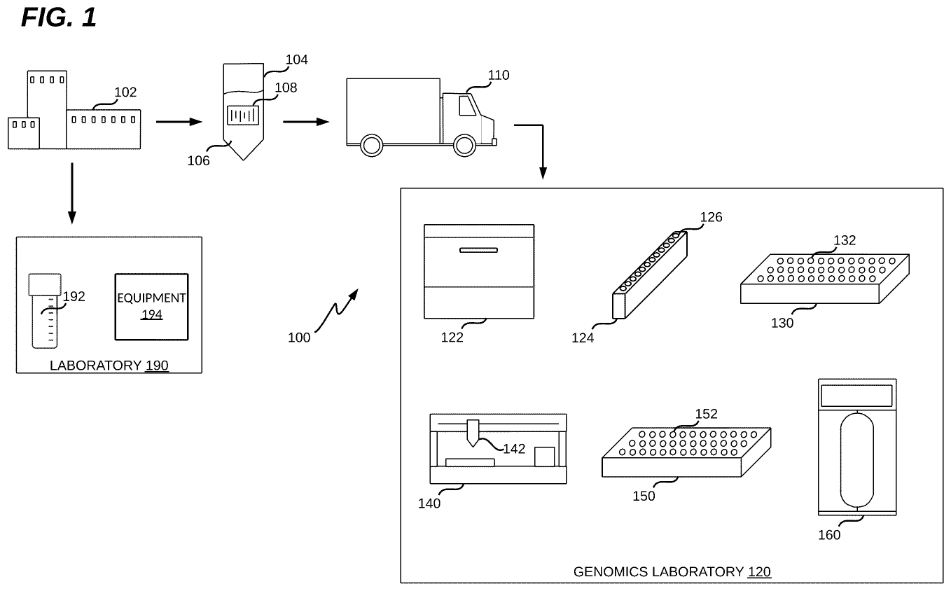

FIG. 1 is a diagram depicting a sample processing architecture 100 in an illustrative embodiment. Sample processing architecture 100 comprises any system or organizational structure for acquiring and sequencing biological samples in a high-volume, high-throughput manner. Sample processing architecture 100 may be utilized, for example, to collect and sequence genetic material (in the form of Ribonucleic Acid (RNA) or Deoxyribonucleic Acid (DNA)) found within thousands or tens of thousands of samples 106 daily, via multiple healthcare provider networks 102.

Healthcare provider networks 102 may comprise hospitals, clinics, practitioner offices, laboratories, surgical centers, etc. that engage in or facilitate the practice of medicine. In one embodiment, healthcare provider networks 102 each comprise groups of hospitals that treat millions of patients. As a part of the practice of medicine, healthcare provider networks 102 acquire samples 106 for sequencing. For example, a healthcare provider network 102 may acquire samples 106 as part of a population screening program, as part of medical treatment, etc. The specific amount of sequencing desired for a sample 106 may comprise a selected set of one or more genes, an exome, the entire genome of a patient, etc. The samples 106 are stored in sample containers 104, which may be accompanied by Customer Sample Identifiers (CSIs) 108. A delivery service 110 provides the samples 106 to a genomics laboratory 120 for processing.

Healthcare provider networks 102 may also acquire samples 192 for blood testing (described below). These samples 192 may be provided to laboratory 190 for analysis via equipment 194 (e.g., a chemically treated test strip, biochemical assay, etc.), or may be analyzed by patients via at-home testing methods. Sample processing architecture 100 provides a technical benefit by allowing laboratory 190 and genomics laboratory 120 to specialize in different methods of analysis.

Procedures within genomics laboratory 120 related to genetics may include accessioning, sample plating, storage, extraction, library preparation, enrichment, and sequencing processes. These processes acquire genetic material from a sample 106, separate the genetic material from other constituents, duplicate the genetic material, and quantify the genetic material order to determine a swathe of sequence data, such as an exome or entire genome for a subject (e.g., a human patient, an organelle of a human patient, etc.). Although the procedures discussed herein are specific with regard to one method of sequencing, other techniques may be utilized in accordance with known standards in order to perform sequencing for samples 106. For example, although the techniques discussed herein relate to hybridization capture techniques, amplicon-based techniques may be used.

Accessioning. Accessioning refers to receiving and preparing samples 106 for later laboratory processes. In one embodiment, accessioning includes receiving a batch of samples 106 (e.g., hundreds or thousands of samples 106) from one or more delivery services 110 each day for processing. For example, packages that each include tens or hundreds of samples 106 may be delivered to genomics laboratory 120 via the United States Postal Service (USPS), or a private package carrier.

Each sample 106 may be retained within a sample container 104, such as a five milliliter (mL) test tube. In this embodiment, the sample container 104 is sealed to prevent the sample 106 from being exposed to the environment and also to prevent the sample 106 from co-mingling with other samples 106. For example, the sample 106 may be sealed via a cap that is threaded, glued, press-fit, etc. At the time of delivery, the sample container 104 may further include a remnant of a sampling tool, such as a portion of a swab that was utilized to acquire the sample.

In many embodiments, a CSI 108 for the sample 106 is reported via a component affixed to or integrated with the sample container 104. The CSI 108 uniquely distinguishes the sample 106 from other samples 106 being received. For example, a CSI 108 may uniquely distinguish a sample 106 from other samples 106 in the same batch, other samples 106 received on the same date, other samples 106 received from the same healthcare provider network 102, etc. A CSI 108 may be reported via a barcode label, Quick Response (QR) code label, Radio Frequency Identifier (RFID) chip, or any suitable visual, transmission-generating, or other physical component affixed to or integrated with the sample container 104.

In further embodiments, the sample container 104 is itself sealed within an external container such as a bag (not shown). Using an external container helps to prevent contamination, by ensuring that a technician at the genomics laboratory 120 does not contact biological material from the sample 106 that may exist on an outer surface of the sample container 104. Use of an external container may also be required by law (e.g., Department of Transportation (DOT) guidelines). Use of an external container additionally helps to prevent cross-contamination between samples 106. Furthermore, in embodiments where samples 106 may include blood or a pathogen, an external container provides an additional barrier to protect the health of technicians. The external container may additionally include documentation confirming the CSI 108, information for the subject that the sample was sourced from, and/or information indicating circumstances of sampling. The circumstances of sampling may include, for example, a sampling date, a sampling method, a location that the sample was acquired, a name or title for a person who performed the sampling, and/or additional notes.

In this embodiment, the sample 106 comprises a chemical solution. For example, the sample 106 may comprise a prepared aqueous solution such as a saline solution, or may comprise a bodily fluid such as blood, saliva, mucus, etc. In some embodiments each of the samples 106 fills between two and five milliliters of volume within its corresponding sample container 104.

The samples 106 further include genetic material such as Deoxyribonucleic Acid (DNA), Ribonucleic Acid (RNA), etc. In many instances, the genetic material is one of many constituent components within the sample 106. For example, the genetic material may exist within the nuclei of white blood cells that are included within the sample 106. In a further example, genetic material may exist within viruses or bacteria within the sample 106. In this embodiment, the genetic material is not yet isolated from the remaining constituent components of the sample 106.

After receipt of the samples 106, batches of the samples 106 (e.g., as stored within sample containers 104 and/or external containers) may be heated in ovens 122 to facilitate cell lysis. The temperature, and duration of heating, may be chosen such that pathogenic material within the samples 106 is rendered harmless, or such that cellular lysis occurs. For example, heating may occur at a temperature of between forty and eighty (e.g., fifty) degrees Celsius (C), for a period of time between fifteen and two hundred (e.g., thirty) minutes. In some embodiments, including embodiments wherein the samples 106 are primarily the contents of a blood draw, the heating step may be foregone.

In this embodiment, upon completion of heating, the batches of samples 106 are removed from the ovens 122. In one embodiment, sample containers 104 are removed from corresponding external containers, such as by cutting the external containers open. With the sample containers 104 now available for direct interaction, the sample containers 104 are inspected. As a part of this process, a technician or automated system may determine the CSI 108 for the sample 106, and may compare the CSI 108 to a CSI 108 listed on documentation provided in the external container. If there is a discrepancy between the CSI 108 on the sample container 104 and a CSI 108 listed in the documentation, the sample 106 may be flagged as having an error condition. Similarly, if the CSI 108 on the sample container 104 is damaged (e.g., abraded, heat-damaged, or water-damaged) and has become unreadable, the sample 106 may be flagged as having an error condition.

A technician or automated system may further inspect the contents of the sample container 104, via visual or other methods. If the sample 106 does not include expected constituent component (or is otherwise non-compliant) then the sample 106 is flagged as having an error condition. For example, if the sample 106 is primarily saliva and includes a fluid that is not permitted (e.g., blood), includes an entire swab or no swab, appears to have a fractured or broken casing, or is outside of an expected range of volume (e.g., between two and five milliliters), then the sample 106 may be flagged as having an error condition.

Samples 106 that have not been flagged as having an error condition proceed to sample integration. In one embodiment, as a part of sample integration, the sample 106 is assigned a Laboratory Sample Identifier (LSI). The LSI uniquely identifies the sample 106 from other samples 106 received for the batch, received on the same day, processed in the same laboratory, and/or handled by the same organization performing sequencing. In many embodiments, the LSI is stored in a memory of a genomics server (e.g., within a laboratory sample database), and is uniquely associated with a corresponding CSI 108 for the sample. The LSI may also be associated with any error conditions reported for the sample 106.

In many embodiments, CSIs 108 originally provided with the samples 106 are in the form of a paper barcode. In such embodiments, the paper barcode may be printed in aqueous ink. This renders the barcode subject to degradation upon exposure to liquid in the laboratory environment, which is undesirable.

To ensure that each sample container 104 is capable of traveling through the genomics laboratory 120 without its identifier being physically degraded, a corresponding LSI may be indicated at the sample container 104. The LSI may be indicated via the application of a barcode label, Quick Response (QR) code, Radio Frequency Identifier (RFID) chip, or other visual, transmission-generating, or other physical component affixed to or integrated with the sample container.

In one embodiment, the LSI is printed onto a barcode label comprising rip-proof material (e.g., vinyl) in a water-insoluble ink. This implementation ensures that the barcode label is resistant to physical and chemical degradation. The barcode may be applied around an entire perimeter of the sample container 104, ensuring that the sample container 104 may be scanned from any angle.

In further embodiments, the element used to report the LSI is accompanied by a visually distinct mark that enables rapid confirmation by a technician that the sample 106 has been integrated into the laboratory environment. The visually distinct mark may comprise a colored ring (e.g., around an entire perimeter of the sample container), a logo, a physical feature, a stamp, etc.

Sample Plating. With the samples 106 having been successfully integrated into the environment of the genomics laboratory 120 environment, the samples 106 are ready for analytics to be performed. To this end, the samples 106 are prepared for transfer to a sample microplate 130. The sample microplate 130 may be labeled with a unique identifier via similar techniques to those used for sample containers 104 above. The unique identifier distinguishes the sample microplate 130 from other sample microplates 130. In one embodiment, the sample microplate 130 comprises a solid body defining three hundred and eighty-four wells, distributed across sixteen rows and twenty-four columns, each well having a capacity of between thirty and one hundred microliters. In a further embodiment, the sample microplate 130 comprises a solid body defining ninety-six wells, distributed across eight rows and twelve columns, each well having a capacity of between one hundred and three hundred microliters. Any suitable number and arrangement of wells may be selected as a matter of design choice.

As a part of preparing the samples 106 for transfer to the sample microplate 130, a technician may place sample containers 104 onto a rack 124, and scan each sample container 104 to determine an LSI for each location 126 (e.g., each container receptacle) on the rack 124. In some embodiments, the rack 124 is assigned a unique identifier that distinguishes it from other racks 124. The rack 124 may be labeled with a unique identifier using techniques similar to those used for sample containers 104. The technician, or automated machinery such as a server operating an optical scanner, may then associate the unique identifier for the rack 124, along with the locations 126 assigned to the samples 106, with the corresponding LSIs of the samples 106 stored at the rack 124.

The technician additionally unseals the sample containers 104. Unsealing of sample containers 104 may be a deeply labor-intensive process, particularly when laboratory processes are performed at scale to handle tens of thousands of samples 106 per day. Thus, a technician may utilize automated tooling to enhance the speed at which sample containers 104 are unsealed. The tooling may, for example, unscrew, cut, or drill each sample container 104, in order to make the sample 106 within available for physical transfer to the sample microplate 130.

One or more racks 124 of samples 106 are provided to a Liquid Handler (LH) 140, such as an automated robot that operates an end effector 142 in accordance with one or more Numerical Control (NC) programs to transfer liquids between wells via arrays of micropipettes. An LH 140 is also known as a “Liquid Handling System.” LH 140 may comprise, for example, a Hamilton Microlab Star Liquid Handling System.

In this embodiment, the LH 140 proceeds to transfer a portion of each sample 106 at a rack 124 to a well 132 within the sample microplate 130 that is not shared with other samples 106. For example, the well 132 for each sample 106 may be predetermined in accordance with a control program used by the genomics laboratory 120. In one embodiment, the LH 140 transfers the portions of the samples 106 to the wells 132 of the sample microplate 130 by providing instructions to actuators, piezoelectric elements, and/or pressure systems operating the end effector 142. In such an embodiment, the end effector 142 may align its array of micropipettes with the sample containers 104 to retrieve portions of the samples 106. Furthermore, in such an embodiment, the end effector 142 may dynamically align its array of micropipettes with the sample microplate 130 to deposit the portions of the samples 106 at the wells 132.

Because there is a known relationship between locations 126 at the rack 124 and wells 132 of the sample microplate 130 (e.g., as indicated by row and column), contents of the memory of a genomics server (e.g., a laboratory sample database) may be updated to indicate the well 132 storing genetic material for each sample 106. In one embodiment, the memory is further updated to associate a unique identifier for the sample microplate 130 with the samples 106 stored therein.

In one embodiment, programmed instructions for the LH 140 may direct the end effector 142 to position itself above a set of disposable tips, descend into the tips to attach the tips, reposition the end effector 142 above the rack of sample containers 104, adjust spacing between micropipettes within the array, descend until the tips reach the sample containers 104, draw liquid from the sample containers 104, deposit the liquid into a well at the sample microplate 130, and then dispose of the tips. Such a process may be repeated across sample containers 104 stored on multiple racks until the sample microplate 130 is filled with portions from the samples 106. In one embodiment, one or more wells 132 on the sample microplate 130 are filled with a control reagent instead of a portion of a sample 106.

The amount of liquid drawn from each sample container 104 may comprise a small fraction of the overall volume of the sample container 104. For example, an amount of liquid drawn may comprise several microliters, such as between two and ten microliters. Upon completion of transfer from the sample containers 104 to the wells, the sample microplate 130 may be covered with a liquid and/or gas-impermeable layer, such as foil or paraffin. Sample containers 104 remaining on the racks may be resealed, for example with pressure-fit caps having a color distinct from an original color for the sample containers. With accessioning now complete for the sample microplate 130, the sample microplate 130 is transferred to a next section of the laboratory for processing.

### Storage

In one embodiment, accessioned samples 106, samples 106 ready for analytics, and/or samples 106 that have already been sequenced, are stored for later use. For example, samples 106, sample containers 104, and/or sample microplates 130 may be stored at room temperature, or may be cryogenically frozen at a low temperature (e.g., negative eighty degrees Celsius) and arranged in racks for later retrieval. Samples 106 may be preserved for periods of days or years, enabling rapid re-testing to be performed for subjects without the need for re-acquiring genetic material. Storage of the samples 106 provides notable value in the event that contents of a well 132 used for sequencing do not meet with rigorous quality control standards. Specifically, storage enables re-sampling to occur in the event that there is a desire to re-sequence a sample 106.

Extraction. Sample microplates 130 are transferred to a portion of the genomics laboratory 120 dedicated to extraction of the genetic material. The segment of the laboratory 120 that performs extraction and other pre-amplification operations may be sealed from, and/or positively pressurized relative to, other portions of the genomics laboratory 120.

During extraction, a sample microplate 130 is acquired and provided to an LH 140. The LH 140 that performs extraction may be different from the LH 140 that performs sample plating. The LH 140 may apply a reagent to each well 132 that lyses cells within each well. For example, this may be performed in order to lyse white blood cells containing genetic material for a human, or may comprise lysing other types of cells to expose other types of genetic material. The reagents used for pre-amplification processes may be stored at the LH 140 in a temperature-controlled manner, and may even be vibrated or mixed on a regular basis to ensure that the reagents are evenly distributed in suspension.

In one embodiment, extraction further includes an LH 140 aspirating and dispensing reagents that selectively bind to genetic material released from the lysed cells. This process may include applying a bead (not shown) to the well 132. In one embodiment, the beads comprise magnetic beads that selectively bind to the genetic material (e.g., DNA). This allows for isolation and purification of the genetic material while contaminants remain in solution. In one embodiment, the magnetic bead is drawn to a magnetic base at or under the sample microplate 130. After the genetic material has been drawn to the bead, and after the bead has been secured to the base of the well, a flushing step may be performed wherein remaining fluid in each well is washed away. This ensures that potential impurities are removed from the well. The LH 140 may further add or remove fluid from each well 132 to perform additional concentration and/or elution of the genetic material, and may transfer fluid from the wells 132 of the sample microplate 130 to wells 152 of a genome stock microplate 150. The genome stock microplate 150 may be labeled with a unique identifier, and the contents of each well 152 of the genome stock microplate 150 may be associated with a corresponding LSI. In all phases of operation, the LH 140 is operated to ensure that fluid is not transferred between wells 152, as this results in contamination.

In one embodiment, a portion of fluid is removed from each well 152 of the genome stock microplate 150 for quality control purposes. Concentration of genetic material within the wells 152 may be confirmed via testing of this fluid, such as by application of a dye that reacts with the genetic material at known levels of fluorescence for known concentrations.

Library Preparation. After extraction is completed, library preparation may be performed for the contents of the genome stock microplate 150. The bead for each well, including ionically bonded genetic material, is transferred to a distinct well of a library preparation microplate (not shown). The library preparation microplate includes an identifier that uniquely distinguishes it from other library preparation microplates, and the LSI associated with each well on the genome stock microplate 150 may be mapped to a corresponding well on the library preparation microplate.

The library preparation microplate may be transferred to a new portion of the genomics laboratory 120 that is sealed from, and/or positively pressurized relative to, other portions of the genomics laboratory 120 that do not perform amplification of genetic material. This feature helps to prevent amplified genetic material from entering portions of the laboratory where genetic material has not been amplified, which could result in contamination. The transfer process may be performed by placing a library preparation microplate into an airlock at the pre-amplification portion of the genomics laboratory 120, sealing the airlock, and then retrieving the library preparation microplate from the airlock via the amplification portion of the genomics laboratory 120.

In one embodiment, a reagent is applied to each well of the library preparation microplate. The reagent ionically bonds to the surface of the bead within the well, and does so more strongly than the genetic material. This releases the genetic material from the surface of the bead of each well, enabling the genetic material to be chemically interacted with.

Library preparation may include normalization of a concentration of genetic material in each well of the library preparation microplate. Library preparation further includes fragmentation of the genetic material via an enzyme or via the application of physical forces. During this process, the entire genome (e.g., roughly three billion base pairs for a human genome), may be fragmented into pieces. In one embodiment, the pieces vary between three hundred and four hundred base pairs in length. These pieces are known as nucleic acid fragments.

In this embodiment, the nucleic acid fragments undergo adaptor ligation and indexing in accordance with known techniques. For example, this may comprise Next Generation Sequencing (NGS) library preparation processes defined by Illumina. Next, a limited amount of Polymerase Chain Reaction (PCR) amplification is performed upon the library. The resulting solution is then purified and eluted via operation of an LH 140.

During library preparation, one or more reference samples of genetic material, distinct from the genetic material found in the samples, may be added to wells of the library preparation microplate. The reference samples do not include genetic material received from a customer, but rather include known sequences of base pairs. The reference samples serve as controls to ensure that processes are carried out with sufficient quality.

Upon completion of library preparation, desired fragments of the genetic material (e.g., thousands or millions of distinct fragments of the genetic material, each corresponding with a different portion of a genome of the subject) have been ligated to predefined adapters (e.g., DNA adapters) that bind with the genetic material. Each of the adaptor-ligated fragments is referred to as a “library.”

In further embodiments, the probes applied to each well of the library preparation plate include chemical identifiers (colloquially referred to as “barcodes”) that are distinct from each other. The use of a different chemical identifier for probes applied to each well of the library preparation microplate enables sequencing to later be performed for multiple subjects on the same flow cell, without conflating sequencing results for those subjects.

The library preparation process may further comprise controlling a concentration of the genetic material in each well, and purification and/or elution of the resulting material. Similar to the processes performed after extraction of genetic material, concentration of genetic material after library preparation may be confirmed for each well via testing.

Enrichment. After library preparation, enrichment processes may be performed in order to either directly amplify (e.g., via amplicon or multiplexed PCR) or capture (e.g., via hybrid capture) predefined libraries. This enhances the case of sequencing desired portions of the genome.

In one embodiment, during enrichment, customized biotinylated oligonucleotide probes are applied to the libraries. The probes selectively hybridize genetic material occupying desired portions of the genome for the genetic material, such as specific genes, or the entire exome. Magnetic beads bind to biotin molecules in the probes to attach the hybridized material to the magnetic beads. Magnetic forces capture the beads in place, enabling remaining fluid within each well to be removed or washed out, thereby removing impurities and leaving only the genetic material that is desired. Genetic material may be released from the beads in a similar manner to that discussed above for prior processes.

In a further embodiment, hybrid capture target enrichment is performed. During this process, the probes comprise tailored oligonucleotides that are chosen to bind to the genetic material. The range of probes may be tailored as a group to bind to specific alleles, specific genes, the exome, the entire genome, etc. That is, each probe may bind to a nucleic acid fragment at a specific location on the genome, and the range of probes may be selected to ensure that alleles, genes, the exome, or the entire genome of the subject being considered is acquired. Utilizing probes in this manner may enhance efficiency of the sequencing process, by foregoing the need to sequence all of the roughly three billion base pairs found in the human genome.

The enrichment process may further comprise controlling a concentration of the genetic material in each well, and purification and/or elution of the resulting material. Similar to the processes performed after extraction of genetic material, concentration of genetic material after enrichment may be confirmed for each well via testing.

Sequencing. Sequencing may be performed according to any of a variety of techniques, including short-read and long-read techniques, via sequencing equipment 160 (e.g., an Illumina NovaSeq X sequencing machine). As provided herein, emphasis will be placed upon short-read sequencing technologies, which are expected to benefit the most from the following methods and techniques. As used herein, short-read sequencing refers to sequencing technologies that generate reads of less than five hundred base pairs in length. Short-read sequencing may be used as the basis for “synthetic long read” technologies that stitch individual short reads together, but as used herein, short-read sequencing does not refer to the creation or use of synthetic long reads.

In one embodiment, short-read sequencing is performed as Sequencing by Synthesis (SBS). For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers referred to above. In one embodiment, the chemical identifiers comprise nucleotide sequences that are detectable during the sequencing process to determine a corresponding LSI.

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flow cell.

In this embodiment, sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence which can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Additionally in this embodiment, base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome, or another set of genomic data, is being tested, different reagents (e.g., probes, primers, etc.) may be chosen. That is, different reagents may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

Bioinformatics. Sequencing data may be stored in any suitable format. In one embodiment, raw sequencing data generated during synthesis is stored in a file format such as Binary Base Call (BCL). This raw data may be fed to an analytical pipeline such as a cloud-based computing environment. Raw sequencing data may be processed by the pipeline into a second format, such as a text-based FASTQ format, that reports quality scores. The second format may then be analyzed to perform alignment of sequence reads to a reference genome, such as a reference genome reported in a Browser Extensible Data (BED) file. The aligned sequence data may be reported as a Binary Alignment Map (BAM) file or Compressed Reference-oriented Alignment Map (CRAM) file. The aligned sequence data may then be called, resulting in a Variant Call Format (VCF) file reporting called variants at each location of the genome that was sequenced, together with secondary metrics such as quality indicator metrics. As used herein, a variant comprises a unique combination of genetic information, in the form of consecutive base pairs at a specific set of locations (e.g., genomic coordinates) along a portion of a chromosome. Each variant is distinguished from other variants by having a different combination of base pairs along the set of locations. This may be due to Single Nucleotide Polymorphisms (SNPs) which relate to common single nucleotide changes, Single Nucleotide Variants (SNVs) which relate to rare nucleotide changes, small variants, insertions and/or deletions (Indels) which relate for example to the insertion or deletion of less than thirty base pairs, or differing numbers of repetitions, Copy Number Variants (CNVs), which relate to larger insertions or deletions, translocations, inversions, other types of genetic variants, or even combinations of variants, such as haplotypes or Multi-nucleotide variants (MNVs).

The called sequence data may be provided to a data analyst via a User Interface (UI), such as a Graphical User Interface (GUI) presented via a display. The technician may then validate the resulting called sequence data and release it for reporting to subjects, health care providers, and/or scientists.

### Genomics Architecture.

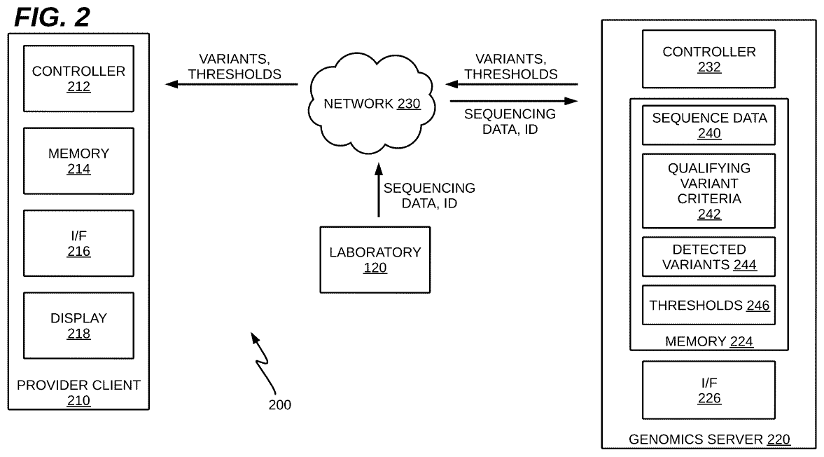

FIG. 2 is a block diagram illustrating a genomics architecture 200 in an illustrative embodiment. Genomics architecture 200 comprises any combination of systems and devices operable to review, process, and/or control access to sequencing data, including sequencing data received from genomics laboratory 120. In this embodiment, genomics architecture 200 comprises a genomics server 220 which receives sequencing data and identifiers (e.g., CSIs 108, LSIs, etc.) from genomics laboratory 120, via network 230.

Genomics server 220 receives the sequencing data via interface (I/F) 226, such as an Ethernet interface, wireless interface compliant with Institute of Electrical and Electronics Engineers (IEEE) 802.11 standards, or other physical interface capable of transmitting and receiving digital data. The sequencing data 240 is stored in memory 224 for the population of patients (e.g., millions of patients) that have been sequenced by laboratory 120, and may be maintained in any suitable format. Examples of such formats include CRAM, VCF, BAM, and others. Memory 224 may store, for example, sequence data 240 describing multiple patients, and this sequence data 240 may be maintained in a de-identified format to facilitate the advancement of research. Memory 224 may be implemented via a cloud storage service, or may comprise a storage medium such as a hard disk or flash memory device.

Memory 224 additionally stores qualifying variant criteria 242, detected variants 244, and thresholds 246 for diagnosis and/or treatment of Lp(a) levels. In one embodiment, the portion of memory 224 storing these components is distinct from the portion of memory 224 storing sequence data 240.

Controller 232 manages the operations of genomics server 220, and may for example analyze sequence data 240 to identify detected variants 244, control access and authentication related to sequence data 240, communicate with one or more provider clients 210, and/or perform additional operations. Controller 232 may be implemented, for example, as custom circuitry, as a hardware processor executing programmed instructions, as a combination of shared hardware processing resources implementing a compute service, or some combination thereof.

Genomics architecture 200 further comprises provider client 210, which is configured to receive information regarding detected variants 244 and/or thresholds 246. In this embodiment, provider client 210 includes a controller 212, a memory 214, an interface (I/F) 216, and a display 218. Controller 212 manages the operations of the provider client 210, and may be implemented, for example, as custom circuitry, as a hardware processor executing programmed instructions, or some combination thereof. Memory 214 comprises information for interpreting the data received via I/F 216. Display 218 may comprise a projector, screen, etc. for presenting information to a user of provider client 210.

Interpreting LPA Sequencing Data. After sequencing data for the patient has been acquired (e.g., as an accompaniment to standard blood testing, in a prior event that provided a sample 106, etc.), sequencing data for the gene LPA is reviewed for the patient by controller 232 of genomics server 220. LPA encodes apo(a), which is a part of Lp(a). LPA resides on chromosome six, has a cytogenetic location of 6q25.3-q26, and genomic coordinates of (GRCh38): chr6:160,531,482-160,664,275.

Two copies of the gene LPA may be expected for most individuals (i.e., one copy of LPA for each copy of chromosome six). However, within LPA, there is tremendous variation of copy number within regions known as kringle-type domains. For example, between two and forty-three copies of kringle-type domains may be common across the general population.

The masking processes described in one or more embodiments of the methods discussed below take into account the high similarity between various portions of LPA, especially kringle IV (“KIV”) domains. By intentionally masking specific portions of LPA within KIV that have high similarity, a diagnostically-relevant pseudocount of copy number, especially for KIV-2, may be determined. Methods 300-500 discuss various methods of beneficially creating and utilizing such information.

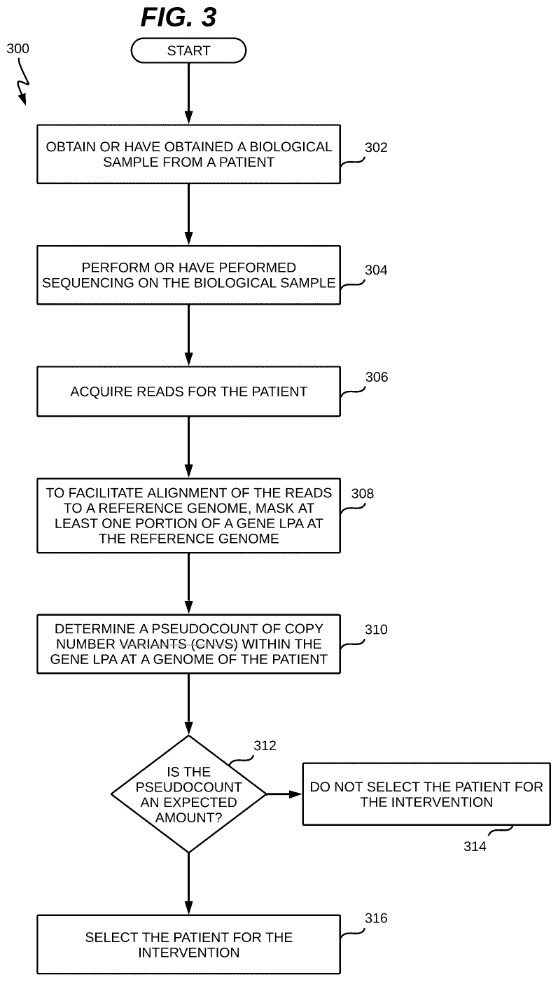

FIG. 3 is a flowchart depicting a method 300 of selectively treating a patient based on a pseudocount of copy number within LPA for the patient. Specifically, method 300 discusses selecting a patient for intervention relating to Lp(a) levels, based on a pseudocount of copy number determined for LPA.

Step 302 includes obtaining or having obtained a biological sample from the patient. In one embodiment, this comprises genomics laboratory 120 acquiring a sample 106 consisting essentially of blood, saliva, cells (e.g., cells acquired via a buccal swab as described above with regard to FIGS. 1-2, or may even include solutions of extracted DNA.

Step 304 includes performing or having performed sequencing (e.g., short-read sequencing, such as SBS) on the biological sample, and may be performed in a similar manner as described above with regard to genomics laboratory 120, sequencing equipment 160, and/or genomics server 220 of FIGS. 1-2.

Step 306 includes acquiring reads for the patient. This may be performed by genomics server 220 acquiring raw sequencing data as that data is generated (e.g., as a Binary Alignment Map (BAM) slice), or by acquiring sequencing data that has been derived from raw sequencing data. In one embodiment, the sequencing data is raw sequencing data in FASTQ format, in a further embodiment, the sequencing data is aligned data in a BAM or CRAM format. For example, raw reads in a FASTQ file may be aligned directly to a masked chr6, or reads that have been aligned to LPA from an alignment BAM/CRAM file may be aligned back to masked chr6. In any case. before aligning reads to masked chr6, all possible reads that could be aligned to LPA are retrieved.

Reads may be acquired for multiple patients in parallel or sequentially, as part of a bioinformatics pipeline that processes sequencing data received from a laboratory. For example, reads may be acquired in batches, as batches of patients are sequenced at the genomics laboratory 120.

Step 308 includes, to facilitate alignment of the reads to a reference genome before or during alignment (e.g., GRCh38), masking at least one portion of a gene LPA at the reference genome. Masking may comprise preventing reads from being aligned to the at least one portion of the gene LPA at the reference genome. Masking has the effect of forcing reads that could otherwise be mapped to regions sharing highly similar sequences.

In one embodiment, controller 232 performs masking that is focused upon the kringle-IV (KIV) domain of LPA. KIV itself includes multiple regions. These range from KIV-1 to KIV-10. Up to seventy percent of the length of the coding portion of LPA consists of KIV-2, which is hypervariable. Thus, masking helps regions which are highly similar to the KIV-2 domain to be mapped to the KIV-2 domain.

On reference genome GRCh38, exons 3-16 of LPA (proceeding from telomere to centromere) cover KIV-1 through KIV-3. In one embodiment, controller 232 engages in masking that is focused upon these exons. For context, masking is a process related to alignment during sequencing. During alignment, reads are aligned to portions of a reference genome (e.g., GRCh38). Reads are aligned to the portion of the reference genome that they most accurately match. The act of masking makes it so that reads are explicitly prevented from being permitted to align with masked portions of the reference genome. Thus, reads which would otherwise map to these portions will be mapped to other portions of the reference genome, or will be discarded. The end result (i.e., mapping to another portion of the reference genome, or discarding) depends on how accurately a read matches the remaining, unmasked portions of the reference genome.

In one embodiment, the masking comprises controller 232 masking chromosome six at the following locations: 160611053-160640063, and 160646511-160646865. The masking may further or alternatively comprise masking exon 3 of the gene LPA, exons 6-16 of the gene LPA, and portions of introns that share high sequence similarity with introns of KIV-2.

In an illustrative embodiment, the following regions are masked, wherein the positive direction proceeds from the telomere to the centromere, and “b” refers to a unit of one nucleotide base. All Exons are padded by 10b both upstream and downstream (i.e., defined as being 10b wider in each direction than expected in the reference genome), prior to masking. The first masked region includes Exon 3, extending from −460b prior to Exon 3 to +594b past Exon 3. The second masked region includes Exons 6-16, from −594b prior to Exon 6 to +500b past Exon 16. The alignment tool used during this process may comprise a Burrows-Wheeler Aligner (BWA) algorithm, or any other suitable alignment tool.

Step 310 includes determining a pseudocount of copy number of KIV-2 within the gene LPA at a genome of the patient. In one embodiment, determining the pseudocount comprises determining a total number of reads for the patient that align with KIV-2 after masking has been performed.

In one embodiment, the pseudocount is the total number of reads (e.g., short reads) for the patient that are aligned with KIV-2 after masking. In a further embodiment, the pseudocount is derived from the total number of reads aligned with KIV-2 after masking, and as such may be an estimated number of copies of KIV-2, or another metric that is not total number of reads. For example, the total number of reads aligned with KIV-2 after masking for an individual patient may be compared to a distribution of total read counts that align with the same region(s) across members of a population, such as the general population or the general population of patients at hospital networks. In short, a pseudocount may comprise a total number of reads that align with KIV-2 for a patient after masking, an estimated count of copy number based on a comparison of population data indicating copy numbers at KIV-2 to population data indicating read counts aligned with KIV-2 after masking (e.g., for other members of the population that have been sequenced using the same assay as the patient, and with the same bait, etc.

Distributions of Lp(a) levels in relation to LPA copy number counts may be population-specific. As such, the population may comprise members having one or more shared demographics to the patient being considered (e.g., patients of a shared ancestry, such as European, African, South Asian, patients of a shared sex assigned at birth, etc.).

A pseudocount does not directly count a number of copies. For many or even all short-read technologies, KIV-2 copy number cannot be directly determined. In embodiments wherein short-read technologies are used for sequencing in step 304, the process of method 300 is capable of identifying individuals having two alleles of LPA with low KIV-2 copy number; this is important for diagnostic insights.

In short, when using short-read whole exome sequencing data, exact KIV-2 copy numbers for both alleles may be challenging to determine. However, a quantification of the total KIV-2 copy number difference among individuals sequenced by the same assay (e.g., a pseudocount) can still be used as a numerical variable to stratify a population by Lp(a) risk.

In one embodiment, it is desirable to know both the total number of reads aligned to KIV-2, as well as the number of reads aligned to other KIV domains. For example, this process may be desirable when considering normalized coverage rather than the raw coverage itself, to get the pseudocount. Normalization may be beneficial, as raw coverage varies depending upon the yield of a given sample. For example, normalization of coverage may be performed based on the total yield for the sample, yield for chromosome 6 for the sample, or other KIV domains for the sample. Since these KIV domains are more similar to KIV-2, and each of these KIV domains may have a similar number of probes targeting them, the other KIV domains are expected to be subjected to similar sequencing artifacts. Consequently, normalizing to other KIV domains should result in smaller amounts of variance. In one embodiment, calculating the normalized coverage of reads is based on a statistical spread of total numbers of reads aligned with the KIV-2 region, for persons within a population that the patient belongs to.

Next, controller 232 determines whether or not the pseudocount is an expected amount. The expected range of pseudocounts may be calculated based on the empirical distribution of pseudocount for members of a population that the patient belongs to. This may comprise sequencing results that have been run on the same assay (e.g., using the same sequencing baits) as was used to sequence the patient, sequencing results for patients sharing a demographic (e.g., ancestry) with the patient, etc. In a further embodiment, the expected amount is set equal to a copy number, or range of copy numbers, that correspond with low risk.

If the patient has a pseudocount that is not an expected amount, such as a pseudocount corresponding with less than fifteen copies of KIV-2, a pseudocount outside of a range defined as one or two standard deviations of the mean for the population etc., then this may indicate higher risk for coronary artery disease. Low pseudocount may indicate a higher than average level of Lp(a) in the blood, which may increase risk of arterial diseases such as coronary artery disease, stroke, peripheral artery disease (PAD), calcific aortic valve disease, and aortic stenosis, etc. over time. Conversely, high pseudocounts are indicative of mutations which result in lower levels of Lp(a) in the blood, decreasing risk of coronary artery disease. The specific mechanism driving this relationship remains debated, although the relationship is known.

In an event that the pseudocount is not an expected amount (e.g., if the pseudocount is smaller than expected) in step 312, processing continues to step 316. Step 316 comprises selecting the patient for an Lp(a)-related intervention. The intervention may comprise at least one action selected from the group consisting of: halting tobacco use for the patient, investigating familial hypercholesterolemia status for the patient, imaging blood vessels of the patient for plaque build-up, investigation a family history of the patient for early onset cardiovascular events (e.g., Coronary Artery Disease (CAD) in men and women younger than forty-five and fifty-five years, respectively), prescribing a statin to the patient, prescribing a PCSK9 inhibitor to the patient, prescribing niacin to the patient, ordering (and/or performing) an Lp(a) blood test, and/or ordering lipoprotein apheresis for the patient.

Step 314 includes, in an event that the pseudocount is an expected amount, omitting selection of the patient for the intervention. This corresponds with the patient having a low amount of Lp(a) related risk indicated from sequencing data for LPA.

In one embodiment, the sequencing data is used not just to determine a pseudocount for CNVs, but also to determine if the patient has at least one qualifying variant the gene LPA (e.g., via genomics server 220). This will be described in detail below.

In further embodiments, pseudocounts are considered together with other factors, such as the presence of small variants in KIV-2, an overall Genetic Risk Score (GRS) for LPA, etc. Small variant detection in non-KIV-2 regions may be determined, for example, using a Senticon Haplotyper and/or GVCFtyper. Small variant detection within the KIV-2 region may be determined, for example, using a Senticon TNscope tool. Specifically, in some embodiments sequencing data for the patient is further reviewed by inspecting VCF data within the genomic coordinates for LPA, and using a tool such as the Ensembl Variant Effect Predictor (VEP) to determine whether any called variants are expected to inactivate Lp(a). These are referred to as “Loss of Function” (LoF) variants. LoF variants may include base pairs that indicate stop_lost, start_lost, splice_donor_variant, frameshift_variant, splice_acceptor_variant, or stop_gained. Such variants may include frame shift mutations, nonsense mutations, mutations at splice sites, insertions and/or deletions that result in stop codons, and others. Reviewing sequencing data for the patient may further comprise inspecting VCF data, and using the Ensembl VEP to identify coding variants within the gene being considered (i.e., LPA). LoF variants may be expected to be protective, lowering apolipoprotein(a) and Lp(a) levels. Coding variants comprise mutations that alter an amino acid encoded by LPA for apolipoprotein(a), but do not inactivate the Lp(a) molecule as a unit. For example, coding variants may include base pairs, residing in predetermined portions of the genes being considered, that indicate stop_lost, missense_variant, start_lost, splice_donor_variant, inframe_deletion, frameshift_variant, splice_acceptor_variant, stop_gained, or inframe_insertion. Some coding variants result in loss of function, but not all LoF variants are coding variants. Collectively, LoF variants and coding variants for the genes being considered are referred to as “qualifying variants.” In one embodiment, qualifying variants are classified based on the predictions of bioinformatics tools such as Polyphen or Sorting Intolerant from Tolerant (SIFT). Polyphen benign variants may be considered any variants having a Polyphen value less than 0.15, while SIFT benign may be considered any variants having a SIFT value that is greater than 0.05. In a further embodiment, variants having other predicted molecular properties, such as splice site variants, etc. are considered qualifying variants. The combination of criteria used to classify a variant as a qualifying variant is maintained in qualifying variant criteria 242, which is stored in memory 224. In some embodiments, any variants in KIV-2 that are associated with high levels of Lp(a) are considered qualifying variants, regardless of whether existing bioinformatics tools and/or other criteria indicate that the variant is benign. Such variants may then be integrated into a model calculating a genetic risk score, such as a polygenic risk score.

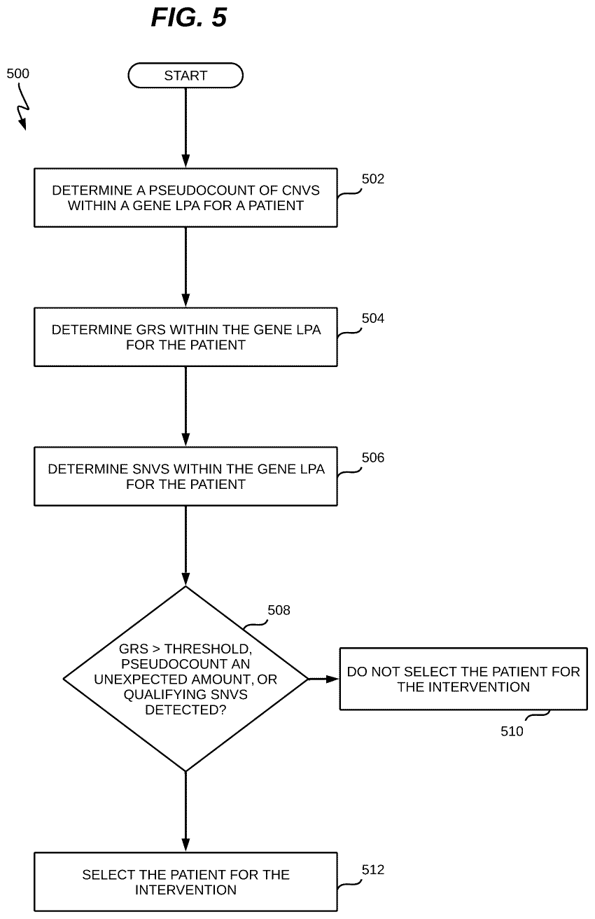

In one embodiment, whenever a qualifying variant is called in a VCF file or similar data structure for a patient, the patient is determined to have a qualifying variant in LPA. In a further embodiment, this determination is made whenever a qualifying variant is confirmed by a variant scientist or automated system. Data on qualifying variants, together with pseudocounts for copy number may be considered in combination before determining whether or not an Lp(a)-related intervention is desirable for the patient. Such techniques are described in further detail with regard to FIG. 5, below.

Method 300 provides a technical benefit by eliminating the need for a specialized, rare, and expensive test (e.g., targeted long-read sequencing, or an Lp(a) blood test), especially whenever short-read sequencing data is available (e.g., for the exome or for LPA specifically). This means that patients who already receive short-read exome sequencing, for example as part of a population screening process for a health network, may be accurately analyzed for the expected amount of Lp(a) in their bloodstream based on genetic data, and hence a concordant amount of cardiovascular risk. Notably, Lp(a) predicts cardiovascular risk independently of LDL. This in turn helps to provide for earlier detection of Lp(a) levels causing increased risk.

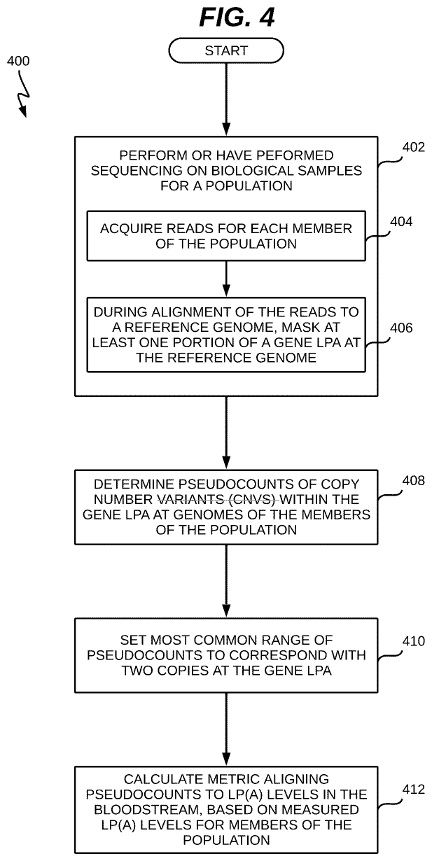

FIG. 4 is a flowchart depicting a method 400 of determining pseudocounts in an illustrative embodiment.

Step 402 includes performing or having performed sequencing on biological samples for a population. Step 404 includes acquiring reads for each member of the population. Step 406 comprises, during alignment of the reads to a reference genome, masking at least one portion of the gene LPA at the reference genome. Steps 402-406 may be performed in a similar manner to steps 304-308 of FIG. 3 above.

Step 408 comprises determining pseudocounts of CNVs within the gene LPA at genomes of the members of the population. This may be determined in a manner similar to step 310 of method 300, and may further comprise determining statistical metrics such as standard deviation, mean, median, mode, and/or variance across the population.

Step 410 comprises establishing the empirical distribution of pseudocounts in a population. In one embodiment, this comprises stratifying the population based on the pseudocounts.

Step 412 comprises calculating a metric aligning pseudocounts to estimated Lp(a) levels in the bloodstream, based on measured Lp(a) levels for the population. In one embodiment, this comprises comparing pseudocount data for patients in a population to Lp(a) levels measured for patients in the population, and then determining a regression formula based on the relationship between pseudocount and Lp(a) level. Lp(a) blood testing data may further help to determine a formula that broadly links pseudocounts to either Lp(a) blood levels, or true CNV counts for the population.

Depending upon preference, one of the following formulae may be used to determine pseudocounts as estimated numbers of copies, wherein CN is copy number, and coverage is a depth of coverage for a given region. Note that KIV-2-1 is the first exon of KIV-2 (exon 4 in the LPA reference sequence in GRCh38), and KIV-2-2 is the second exon of KIV-2 (exon 5 in the LPA reference sequence in GRCh38). The total copy number of KIV-2, estimated through KIV-2 exon 1, is in one embodiment quantified as the following normalized coverage of KIV-2 exon 1, using KIV-i−1 to denote the coverage of KIV-i domain exon 1:

$${CNE}_{KIV - 2 - 1} = KIV - 2 - 1 / ( 1 / 8 \times {\sum }_{i} KIV - i - 1 ) , where \qquad ( 1 )$$

$$i ∈ \{ 1 , 4 , 5 , … , 10 \} ;$$

Similarly, in one embodiment the copy number estimated through KIV-2 exon 2 is quantified as:

$${CNE}_{KIV - 2 - 2} = KIV - 2 - 2 / {( 1 / 8 \times {\sum }_{i = 3}^{10} KIV - i - 1 )}_{/} \qquad ( 2 )$$

In one embodiment the final total copy number of KIV-2 is quantified as:

$${CNE}_{KIV - 2} = 1 / 2 \times ( {CNE}_{KIV - 2 - 1} + {CNE}_{KIV - 2 - 2} ) . \qquad ( 3 )$$

Afterwards, this copy number of KIV-2 is normalized by exome assay version to account for variability in region coverage, forming the final KIV-2 CNE.

Method 400 provides a notable technical benefit by permitting sequencing data, especially short-read sequencing data, to be analyzed in a processing-efficient manner that provides insight into Lp(a) levels for patients on a population scale, especially at high and low ends of the spectrum.

FIG. 5 is a flowchart depicting a method 500 for multi-factor evaluation of genetic risk related to Lp(a) in an illustrative embodiment. Step 502 comprises determining a pseudocount of CNVs within LPA for a patient. This may be performed via the corresponding steps of methods 300 and/or 400 above.

Step 504 comprises determining a Genetic Risk Score (GRS) within LPA for the patient. GRS-predicted Lp(a) may be determined, for example, by the technique described in Trinder M, Uddin M M, Finneran P, Aragam K G, Natarajan P. Clinical Utility of Lipoprotein(a) and LPA Genetic Risk Score in Risk Prediction of Incident Atherosclerotic Cardiovascular Disease. JAMA Cardiol. 2020 Oct. 6; 6(3):1-9. doi: 10.1001/jamacardio.2020.5398. Epub ahead of print. PMID: 33021622; PMCID: PMC7539232.

Step 506 comprises determining small variants within LPA for the patient. This may be performed by assigning weights to given variants, and may be independent of the LoF and coding variant detection processes discussed above. Small variants in KIV-2 may be used in the same way as those in non-KIV-2 regions, i.e., via incorporation into a GRS model.

Step 508 comprises determining if the GRS is greater than a threshold, if the pseudocount is other than an expected amount, or if qualifying small variants are detected. If none of the above conditions are fulfilled, then processing continues to step 510, wherein the patient is categorized as low risk, and is not selected for an Lp(a)-related intervention. Alternatively if one or more of the conditions are fulfilled, then processing continues to step 512, wherein the patient is selected for an Lp(a)-related intervention. In further embodiments, a patient is required to meet two or more of the conditions in order to be selected for intervention. In a still further embodiment, the patient may be required to have a lower-than-expected pseudocount (e.g., less than fifteen copies) and a high-risk GRS within LPA to be selected for an Lp(a)-related intervention in step 512.

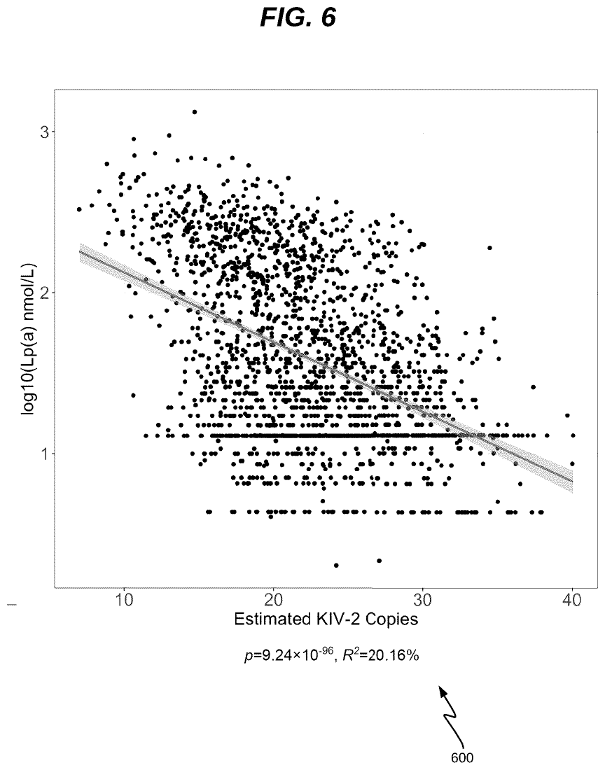

FIG. 6 is a graph 600 that depicts correlations between pseudocounts and measured Lp(a) levels in an illustrative embodiment. FIG. 6 depicts the results of an analysis of a population of roughly one thousand nine hundred and seventeen patients. As shown in FIG. 6, data indicates that the pseudocount, using coverage generated by properly aligned reads, even for short-read technologies, correlate inversely and predictably with measured Lp(a) levels in the blood.

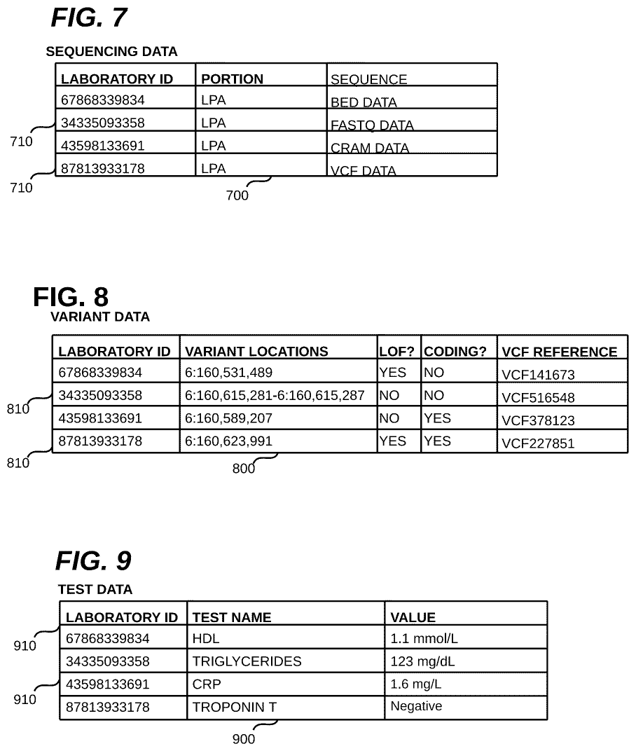

FIG. 7 is a table 700 that summarizes sequencing data for the gene LPA for individuals in an illustrative embodiment. For example, table 700 may be one of many data structures stored in genomics server 220. In this embodiment, table 700 includes an entry 710 for each of multiple patients. Each entry 710 includes a unique identifier (e.g., LSID) for the corresponding patient, as well as an indication of the gene that the sequence data relates to. The portion of the genome that has been sequenced may comprise whole genome data, whole exome data, array data, data for a specific gene or portion of a gene, etc. In this embodiment, the sequence data relates to the gene LPA. Table 700 also indicates a format of the sequence data.

FIG. 8 is a table 800 that summarizes LPA variant data for individuals in an illustrative embodiment. In this embodiment, each entry 810 in table 800 reports a location (e.g., chromosomal coordinate) for each genetic variant of LPA, together with flags indicating whether the variant is LoF or coding variant. Table 800 further includes a VCF reference, which refers to the location and/or identifier of a VCF file that indicates the presence of the variant. Table 800 may be utilized by controller 232 of genomics server 220, in order to rapidly select and report diagnostic and treatment thresholds for a patient.

FIG. 9 is a table 900 that summarizes biomarker test data for individuals in an illustrative embodiment. Specifically, table 900 summarizes test data pertaining to LPA and/or cardiovascular disease for each of multiple patients in an illustrative embodiment. Each entry 910 in table 900 indicates an anonymized laboratory ID for a patient, a corresponding test name, and a corresponding value. Table 900 may be created, for example, based on EHR data retrieved for patients. Laboratory IDs may be associated with EHR identifiers at genomics server 220 or provider client 210, in order to enable access to both health data and genomics data for a patient.

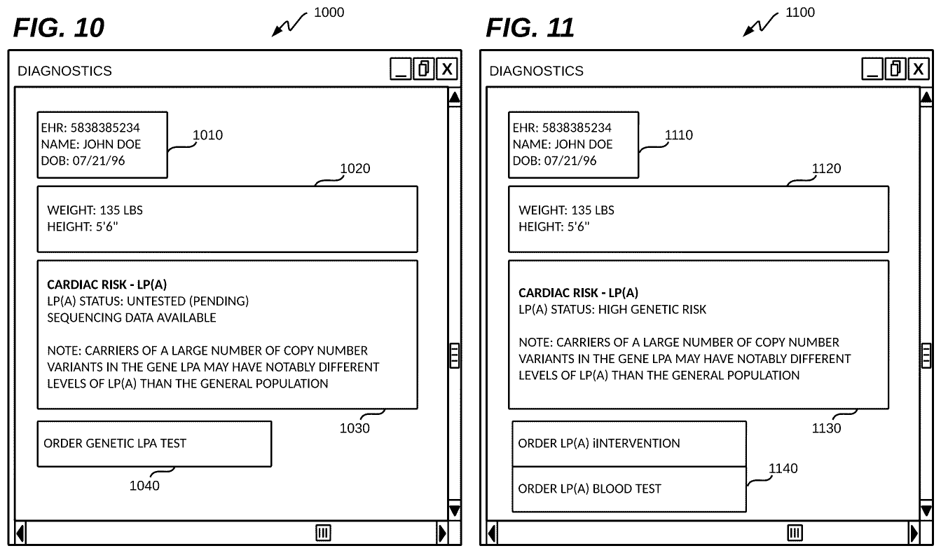

FIGS. 10-11 depict Graphical User Interfaces (GUIs) that facilitate acquisition of LPA risk status, and/or tests for follow-up diagnosis and treatment for a patient having a high LPA risk status, in illustrative embodiments.

FIG. 10 depicts a Graphical User Interface (GUI) 1000 that dynamically recommends sequencing for patients that have an unknown status of risk for the gene LPA in an illustrative embodiment. In this embodiment, GUI 1000 includes region 1010 which provides identifying information for a patient, and region 1020 which depicts phenotypic information for the patient. Regions 1010 and 1020 may be populated, for example, by accessing data within an EHR for the patient maintained at a server accessed by provider client 210 of FIG. 2. Region 1030 provides an indication of whether LPA genetic risk status for the patient is known, such as based on information in an Electronic Health Record (EHR) for the patient. In one embodiment the EHR does not include LPA genetic risk status, and the provider client 210 transmits a message to genomics server 220 to determine whether the patient has sequencing data LPA genetic risk. If genomics server 220 has this sequencing data, a medical practitioner may press button 1040 to order this information from genomics server 220 for instant delivery. Alternatively, if genomics server 220 does not have this sequencing data, a press of button 1040 may trigger an order for a blood draw or saliva sample to be provided to genomics laboratory 120 for sequencing.

FIG. 11 depicts a Graphical User Interface (GUI) 1100 that dynamically recommends additional testing, and/or revised diagnostic or treatment thresholds, for patients who have a high level of LPA genetic risk in an illustrative embodiment indicated by prior-performed sequencing. For example, GUI 1100 may be a variation of GUI 1000 of FIG. 10. Regions 1110 and 1120 may be populated, for example, by accessing an EHR for the patient maintained at a server accessed by provider client 210 of FIG. 2. Region 1130 provides an indication of whether LPA risk status for the patient is known, and may be populated based on data from genomics server 220. In this embodiment, the LPA risk status for the patient is both known and high. Thus, GUI 1100 presents button 1140 for requesting an intervention, such as ordering treatment via lipoprotein apheresis, prescribing estrogen, prescribing niacin, and/or prescribing PCSK9 inhibitors), or further testing (e.g., by ordering an Lp(a) blood test for the patient, by ordering imaging of the heart and/or arteries for plaque build-up, etc.).

Any of the various computing and/or control elements shown in the figures or described herein may be implemented as hardware, as a processor implementing software or firmware, or some combination of these. For example, an element may be implemented as dedicated hardware. Dedicated hardware elements may be referred to as “processors,” “controllers,” or some similar terminology. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, a network processor, application specific integrated circuit (ASIC) or other circuitry, field programmable gate array (FPGA), read only memory (ROM) for storing software, random access memory (RAM), non-volatile storage, logic, or some other physical hardware component or module.

In one embodiment, instructions stored on a computer readable medium direct a computing system of any of the devices and/or servers discussed herein, such as genomics server 220, to perform the various operations disclosed herein. In some embodiments, all or portions of these operations may be implemented in a networked computing environment, such as a cloud computing system. Cloud computing often includes on-demand availability of computer system resources, such as data storage (cloud storage) and computing power, without direct active management by an entity. Cloud computing relies on the sharing of resources, and generally includes on-demand self-service, broad network access, resource pooling, rapid elasticity, and measured service.

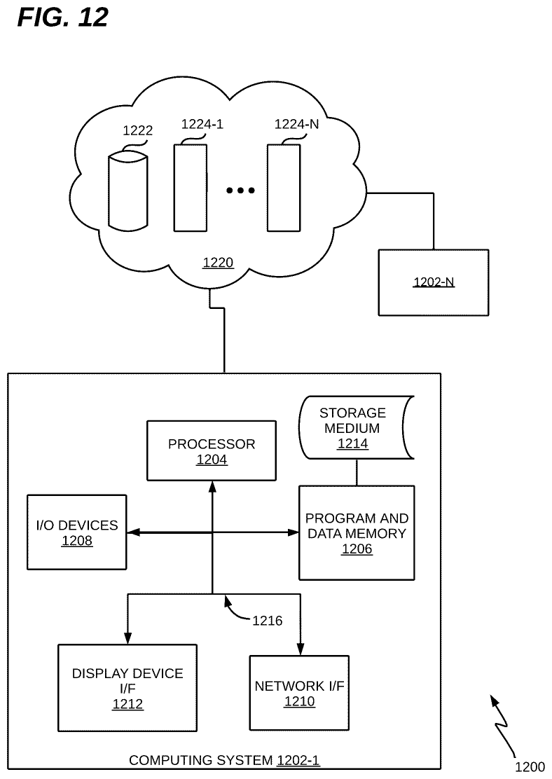

FIG. 12 depicts one illustrative cloud computing system 1200 operable to perform the above operations by executing programmed instructions tangibly embodied on one or more computer readable storage mediums. The cloud computing system 1200 generally includes the use of a network of remote servers hosted on the internet to store, manage, and process data, rather than a local server or a personal computer (e.g., in the computing systems 1202-1-1202-N). Cloud computing enables users to use infrastructure and applications via the internet, without installing and maintaining them on-premises. In this regard, the cloud computing network 1220 may include virtualized information technology (IT) infrastructure (e.g., servers 1224-1-1224-N, the data storage module 1222, operating system software, networking, and other infrastructure) that is abstracted so that the infrastructure can be pooled and/or divided irrespective of physical hardware boundaries. In some embodiments, the cloud computing network 1220 can provide users with services in the form of building blocks that can be used to create and deploy various types of applications in the cloud on a metered basis.

Various components of the cloud computing system 1200 may be operable to implement the above operations in their entirety or contribute to the operations in part. For example, a computing system 1202-1 may be used to perform analysis of gene sequencing data, and then store that analysis along with the gene sequencing data in a data storage module 1222 (e.g., a database) of a cloud computing network 1220. Various computer servers 1224-1-1224-N of the cloud computing network 1220 may be used to operate on the gene sequencing data and/or transfer the gene sequencing analysis and/or the gene sequencing data to another computing system 1202-N.

Some embodiments disclosed herein may utilize instructions (e.g., code/software) accessible via a computer-readable storage medium for use by various components in the cloud computing system 1200 to implement all or parts of the various operations disclosed hereinabove. Examples of such components include the computing systems 1201-1202-N.

Exemplary components of the computing systems 1201-1202-N may include at least one processor 1204, a computer readable storage medium 1214, program and data memory 1206, input/output (I/O) devices 1208, a display device interface 1212, and a network interface 1210. For the purposes of this description, the computer readable storage medium 1214 comprises any physical media that is capable of storing a program for use by the computing system 1202. For example, the computer-readable storage medium 1214 may be an electronic, magnetic, optical, electromagnetic, infrared, semiconductor device, or other non-transitory medium. Examples of the computer-readable storage medium 1214 include a solid-state memory, a magnetic tape, a removable computer diskette, a random access memory (RAM), a read-only memory (ROM), a rigid magnetic disk, and an optical disk. Some examples of optical disks include Compact Disk-Read Only Memory (CD-ROM), Compact Disk-Read/Write (CD-R/W), Digital Versatile Disc (DVD), and Blu-Ray Disc.

The processor 1204 is coupled to the program and data memory 1206 through a system bus 1216. The program and data memory 1206 include local memory employed during actual execution of the program code, bulk storage, and/or cache memories that provide temporary storage of at least some program code and/or data in order to reduce the number of times the code and/or data are retrieved from bulk storage (e.g., a hard disk drive, a solid state drive, or the like) during execution.

Input/output or I/O devices 1208 (including but not limited to keyboards, displays, touchscreens, microphones, pointing devices, etc.) may be coupled either directly or through intervening I/O controllers. Network adapter interfaces 1210 may also be integrated with the system to enable the computing system 1202 to become coupled to other computing systems or storage devices through intervening private or public networks. The network adapter interfaces 1210 may be implemented as modems, cable modems, Small Computer System Interface (SCSI) devices, Fibre Channel devices, Ethernet cards, wireless adapters, etc. Display device interface 1212 may be integrated with the system to interface to one or more display devices, such as screens for presentation of data generated by the processor 1204.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

### ADDITIONAL EXAMPLES

Example 1. A method for selecting a patient for intervention relating to genetically predicted lipoprotein (a) (“Lp(a)”) levels, the method comprising: obtaining or having obtained a biological sample from the patient; performing or having performed sequencing on the biological sample, comprising: acquiring reads for the patient; and masking at least one portion of a gene LPA at a reference genome to facilitate alignment of the reads to a reference genome; determining a pseudocount of copy number within the gene LPA at a genome of the patient; in an event that the pseudocount is not an expected amount, selecting the patient for the intervention; and in an event that the pseudocount is an expected amount, omitting selection of the patient for the intervention.

Example 2. The method of example 1 wherein: determining the pseudocount comprises determining a normalized coverage of reads aligned with KIV domains of the LPA gene at the genome of the patient; and the expected amount corresponds with an expected amount of Lp(a) in blood of the patient.

Example 3. The method of example 2, further comprising calculating the normalized coverage of reads based on a statistical spread of total numbers of reads aligned with a KIV-2 region for persons within a population that the patient belongs to.

Example 4. The method of example 1, wherein: the sequencing comprises short-read sequencing; and the masking comprises preventing reads from being aligned to the at least one portion of the gene LPA at the reference genome.

Example 5. The method of example 4, wherein the masking at least one portion of the gene LPA comprises masking chromosome 6 at locations 160611053-160640063 and 160646511-160646865.

Example 6. The method of example 4, wherein the masking at least one portion of the gene LPA comprises masking exon 3 of the gene LPA, and exons 6-16 of the gene LPA.

Example 7. The method of example 1, wherein the intervention comprises at least one action selected from the group consisting of: investigating familial hypercholesterolemia status for the patient, imaging blood vessels of the patient for plaque build-up, investigation a family history of the patient for early onset cardiovascular events, ordering an Lp(a) blood test for the patient, ordering lipoprotein apheresis for the patient, prescribing a statin to the patient, prescribing a PCSK9 inhibitor to the patient, and prescribing niacin to the patient.

Example 8. A non-transitory computer readable medium embodying programmed instructions which, when executed by a processor, are operable for performing a method for selecting a patient for intervention relating to genetically predicted lipoprotein (a) (“Lp(a)”) levels, the method comprising: obtaining or having obtained a biological sample from the patient; performing or having performed sequencing on the biological sample, comprising: acquiring reads for the patient; and masking at least one portion of a gene LPA at a reference genome to facilitate alignment of the reads to a reference genome; determining a pseudocount of copy number within the gene LPA at a genome of the patient; in an event that the pseudocount is not an expected amount, selecting the patient for the intervention; and in an event that the pseudocount is an expected amount, omitting selection of the patient for the intervention.

Example 9. The medium of example 8, wherein: determining the pseudocount comprises determining a normalized coverage of reads aligned with KIV domains of the LPA gene at the genome of the patient; and the expected amount corresponds with an expected amount of Lp(a) in blood of the patient.

Example 10. The medium of example 9, wherein the method further comprises calculating the normalized coverage of reads based on a statistical spread of total numbers of reads aligned with a KIV-2 region for persons within a population that the patient belongs to.

Example 11. The medium of example 8, wherein: the sequencing comprises short-read sequencing; and the masking comprises preventing reads from being aligned to the at least one portion of the gene LPA at the reference genome.

Example 12. The medium of example 11, wherein the masking at least one portion of the gene LPA comprises masking chromosome 6 at locations 160611053-160640063 and 160646511-160646865.

Example 13. The medium of example 11, wherein the masking at least one portion of the gene LPA comprises masking exon 3 of the gene LPA, and exons 6-16 of the gene LPA.

Example 14. The medium of example 8, wherein the intervention comprises at least one action selected from the group consisting of: investigating familial hypercholesterolemia status for the patient, imaging blood vessels of the patient for plaque build-up, investigation a family history of the patient for early onset cardiovascular events, ordering an Lp(a) blood test for the patient, ordering lipoprotein apheresis for the patient, prescribing a statin to the patient, prescribing a PCSK9 inhibitor to the patient, and prescribing niacin to the patient.

Example 15. A system for selecting a patient for intervention relating to genetically predicted lipoprotein (a) (“Lp(a)”) levels, the system comprising: a genomics server, comprising: an interface configured to acquire reads for a patient; and a controller configured to acquire reads for the patient, and mask at least one portion of a gene LPA at a reference genome to facilitate alignment of the reads to a reference genome, the controller further configured to determine a pseudocount of copy number within the gene LPA at a genome of the patient; and in an event that the pseudocount is not an expected amount, the controller is configured to select the patient for the intervention; and in an event that the pseudocount is an expected amount, the controller is configured to omit selection of the patient for the intervention.

Example 16. The system of example 15, wherein the controller is configured to determine the pseudocount by determining a normalized coverage of reads aligned with KIV domains of the LPA gene at the genome of the patient, wherein the expected amount corresponds with an expected amount of Lp(a) in blood of the patient.

Example 17. The system of example 16, further comprising the controller is configured to calculate the normalized coverage of reads based on a statistical spread of total numbers of reads aligned with a KIV-2 region for persons within a population that the patient belongs to.

Example 18. The system of example 15, wherein the reads comprise short-read sequencing data, and the controller is configured to mask by preventing reads from being aligned to the at least one portion of the gene LPA at the reference genome.

Example 19. The system of example 18, wherein the controller is configured to mask the at least one portion of the gene LPA by masking chromosome 6 at locations 160611053-160640063 and 160646511-160646865.

Example 20. The system of example 18, wherein the controller is configured to mask the at least one portion of the gene LPA by masking exon 3 of the gene LPA, and exons 6-16 of the gene LPA.

Each of these non-limiting examples can stand on its own, or can be combined in various permutations or combinations with one or more of the other examples.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method for selecting a patient for intervention relating to genetically predicted lipoprotein (a) (“Lp(a)”) levels, the method comprising:
obtaining or having obtained a biological sample from the patient;
performing or having performed sequencing on the biological sample, comprising:
acquiring reads for the patient; and
masking at least one portion of a gene LPA at a reference genome to facilitate alignment of the reads to a reference genome;
determining a pseudocount of copy number within the gene LPA at a genome of the patient;
in an event that the pseudocount is not an expected amount, selecting the patient for the intervention; and
in an event that the pseudocount is an expected amount, omitting selection of the patient for the intervention.

2. The method of claim 1 wherein:
determining the pseudocount comprises determining a normalized coverage of reads aligned with KIV domains of the LPA gene at the genome of the patient; and
the expected amount corresponds with an expected amount of Lp(a) in blood of the patient.

3. The method of claim 2, further comprising calculating the normalized coverage of reads based on a statistical spread of total numbers of reads aligned with a KIV-2 region for persons within a population that the patient belongs to.

4. The method of claim 1, wherein:
the sequencing comprises short-read sequencing; and
the masking comprises preventing reads from being aligned to the at least one portion of the gene LPA at the reference genome.

5. The method of claim 4, wherein the masking at least one portion of the gene LPA comprises masking chromosome 6 at locations 160611053-160640063 and 160646511-160646865.

6. The method of claim 4, wherein the masking at least one portion of the gene LPA comprises masking exon 3 of the gene LPA, and exons 6-16 of the gene LPA.

7. The method of claim 1, wherein the intervention comprises at least one action selected from the group consisting of: investigating familial hypercholesterolemia status for the patient, imaging blood vessels of the patient for plaque build-up, investigation a family history of the patient for early onset cardiovascular events, ordering an Lp(a) blood test for the patient, ordering lipoprotein apheresis for the patient, prescribing a statin to the patient, prescribing a PCSK9 inhibitor to the patient, and prescribing niacin to the patient.

8. A non-transitory computer readable medium embodying programmed instructions which, when executed by a processor, are operable for performing a method for selecting a patient for intervention relating to genetically predicted lipoprotein (a) (“Lp(a)”) levels, the method comprising:
obtaining or having obtained a biological sample from the patient;
performing or having performed sequencing on the biological sample, comprising:
acquiring reads for the patient; and
masking at least one portion of a gene LPA at a reference genome to facilitate alignment of the reads to a reference genome;
determining a pseudocount of copy number within the gene LPA at a genome of the patient;
in an event that the pseudocount is not an expected amount, selecting the patient for the intervention; and
in an event that the pseudocount is an expected amount, omitting selection of the patient for the intervention.

9. The medium of claim 8, wherein:
determining the pseudocount comprises determining a normalized coverage of reads aligned with KIV domains of the LPA gene at the genome of the patient; and
the expected amount corresponds with an expected amount of Lp(a) in blood of the patient.

10. The medium of claim 9, wherein the method further comprises calculating the normalized coverage of reads based on a statistical spread of total numbers of reads aligned with a KIV-2 region for persons within a population that the patient belongs to.

11. The medium of claim 8, wherein:
the sequencing comprises short-read sequencing; and
the masking comprises preventing reads from being aligned to the at least one portion of the gene LPA at the reference genome.

12. The medium of claim 11, wherein the masking at least one portion of the gene LPA comprises masking chromosome 6 at locations 160611053-160640063 and 160646511-160646865.

13. The medium of claim 11, wherein the masking at least one portion of the gene LPA comprises masking exon 3 of the gene LPA, and exons 6-16 of the gene LPA.

14. The medium of claim 8, wherein the intervention comprises at least one action selected from the group consisting of: investigating familial hypercholesterolemia status for the patient, imaging blood vessels of the patient for plaque build-up, investigation a family history of the patient for early onset cardiovascular events, ordering an Lp(a) blood test for the patient, ordering lipoprotein apheresis for the patient, prescribing a statin to the patient, prescribing a PCSK9 inhibitor to the patient, and prescribing niacin to the patient.

15. A system for selecting a patient for intervention relating to genetically predicted lipoprotein (a) (“Lp(a)”) levels, the system comprising:
a genomics server, comprising:
an interface configured to acquire reads for a patient; and
a controller configured to acquire reads for the patient, and mask at least one portion of a gene LPA at a reference genome to facilitate alignment of the reads to a reference genome,
the controller further configured to determine a pseudocount of copy number within the gene LPA at a genome of the patient; and
in an event that the pseudocount is not an expected amount, the controller is configured to select the patient for the intervention; and
in an event that the pseudocount is an expected amount, the controller is configured to omit selection of the patient for the intervention.

16. The system of claim 15, wherein the controller is configured to determine the pseudocount by determining a normalized coverage of reads aligned with KIV domains of the LPA gene at the genome of the patient, wherein the expected amount corresponds with an expected amount of Lp(a) in blood of the patient.

17. The system of claim 16, further comprising the controller is configured to calculate the normalized coverage of reads based on a statistical spread of total numbers of reads aligned with a KIV-2 region for persons within a population that the patient belongs to.

18. The system of claim 15, wherein the reads comprise short-read sequencing data, and the controller is configured to mask by preventing reads from being aligned to the at least one portion of the gene LPA at the reference genome.

19. The system of claim 18, wherein the controller is configured to mask the at least one portion of the gene LPA by masking chromosome 6 at locations 160611053-160640063 and 160646511-160646865.

20. The system of claim 18, wherein the controller is configured to mask the at least one portion of the gene LPA by masking exon 3 of the gene LPA, and exons 6-16 of the gene LPA.
